(19) United States
(12) Patent Application Publication
(10) Pub. No.: US 2025/0098987 A1
(43) Pub. Date: Mar. 27, 2025

(54) Breath Signal Estimation Using Point Cloud Data

(71) Applicant: Apple Inc., Cupertino, CA (US)

(72) Inventors: Izzet B. Yildiz, Sunnyvale, CA (US); Aaron Wetzler, Rishon Letzion (IL); Jonathan Hadida, Redwood City, CA (US); Stephen Meisenhelter, San Francisco, CA (US); Kaden J. Strand, Sunnyvale, CA (US); Dimitri E. Diakopoulos, San Jose, CA (US); Grant H. Mulliken, Los Gatos, CA (US)

(73) Assignee: Apple Inc., Cupertino, CA (US)

(21) Appl. No.: 18/890,616

(22) Filed: Sep. 19, 2024

Related U.S. Application Data
(60) Provisional application No. 63/584,836, filed on Sep. 22, 2023.

(51) Int. Cl. A61B 5/113; A61B 5/00; A61B 5/08; A61B 5/11; G06T 7/246; G06T 7/593

(52) U.S. Cl. CPC A61B 5/113; A61B 5/0816; A61B 5/1128; A61B 5/6803; G06T 7/248; G06T 7/593; G06T 2207/10012; G06T 2207/10028; G06T 2207/20021; G06T 2207/30004

(57) ABSTRACT

Respiration data is measured from point cloud data containing points associated with an upper body of a user. A device may receive the point cloud data via a wired or wireless connection or may generate it from one or more image sensors integrated into the device. Movement of points associated with the upper body, or within a region of interest, may be measured to estimate a breath signal. The estimated breath signal may be further processed to derive values representative of the breath signal. The breath signal may be then provided to a given application or location.

### PRIORITY CLAIM

This application claims benefit of priority to U.S. Provisional Application Ser. No. 63/584,836, entitled “Breath Signal Estimation Using Point Cloud Data,” filed Sep. 22, 2023, and which is hereby incorporated herein by reference in its entirety.

### BACKGROUND

Some systems may be improved by being provided physiological information about a user of the system. For example, a health monitoring application may provide informative information to a user if provided physiological measurements related to the user. As another example, an application providing a user an immersive experience, such as a video game, may customize the immersive experience based on a state of the user as determined at least in part based on physiological measurements. Such systems may include a head-mounted device (HMD) such as an extended reality (XR), mixed reality (MR), and/or augmented reality (AR) device.

### SUMMARY

In some embodiments, breath estimation may be performed using three-dimensional volumetric data captured by a mobile electronic device worn or otherwise used by a user. For example, a mobile electronic device comprising sensors with a view of a user's upper body may capture three-dimensional volumetric data and such captured data may be used to estimate a breathing pattern of the user. More specifically, in some embodiments, such methods may be implemented in a head-mounted device (HMD), such as a headset, helmet, goggles, or glasses.

In some embodiments, a breath signal generation system may capture three-dimensional representations of a user's upper body using image sensors, LiDAR sensors, etc. of such devices and this captured data may be used to generate three-dimensional volumetric representations of the user's upper body that change over time as the user breaths. This three-dimensional volumetric data depicting the user's upper body over time may further be analyzed to estimate a breath signal. In some embodiments, other types of devices may be used, such as devices with sensors with a view of at least part of an upper body of a user. For example, cameras included in a desktop computer, laptop computer, tablet, phone, etc. may be positioned such that the sensors have a view of a user's upper body and such cameras may be used to capture three-dimensional volumetric data indicating changes over time with respect to the user's upper body, wherein such changes are analyzed to determine a breath signal. Likewise, as another example, cameras or other sensors of a head-mounted display (HMD) may be positioned with a view of a user's upper body and may similarly be used to capture three-dimensional volumetric data that is used to estimate a breath signal of the user.

In some embodiments, a breath signal generation system may determine a region of interest (ROI) in the captured three-dimensional volumetric data, wherein the ROI encompasses an upper body of a user that may, for example, be used to distinguish portions of the three-dimensional volumetric data that correspond to the user's upper body from other portions of the three-dimensional volumetric data that do not correspond with the user's upper body, such as other portions of the user's body, other objects in view of the sensors, a background, etc. In such embodiments, an identified region of interest in the three-dimensional volumetric data may be analyzed to estimate a breath signal for the user. For example, limiting the analysis to a ROI may reduce an amount of the three-dimensional volumetric data that is analyzed and thus simplify the analysis.

In some embodiments, a ROI may be further divided into two or more subregions that may, for example be used to estimate two or more independent subregion breath signals. A breath signal generation system may use a neural network to generate breath signals, including signals based on subregions of region of interest, from the three-dimensional volumetric data. In some embodiments, the breath signal generation system may combine such subregion breath signals together to estimate a final estimated breath signal for the user which may be more resistant to noise resulting from obstacles close to the user's chest than a single subregion breath signal might be. In some embodiments, different weights may be applied when combining the two or more subregion breath signals into a final breath signal.

In some embodiments, three-dimensional volumetric data may be converted from a local coordinate system to a world coordinate system. For example, three-dimensional volumetric data in world coordinates may be more informative for breath signal generation than three-dimensional volumetric data represented in a sensor-based coordinate system. An example of a world coordinate system may be a coordinate system that is independent of a location of a sensor that captures the points. For example, point A may be defined as being separated from point B by a vector having X, Y, and Z dimensions. An example of a sensor-based coordinate system may be a coordinate system that is based on the position of points relative to sensors. For example, in sensor-based coordinate system both point A and point B may be defined by vectors relative to sensors that captured the respective points. A coordinate system based on the frontal plane of a user, for example, may be a type of world coordinate system. For example, points defined in a relation to frontal plane of the user can be defined without reference to a particular sensor that was used to capture the points. In some embodiments, a breath-signal generation system may use a coordinate system based on the frontal plane of a user's upper body to generate a breath signal. This may allow for comparison of points captured by different sensors, wherein the points captured by different sensors are defined in a shared (e.g., world) coordinate system. In such embodiments, movement of a user's upper body in a direction that is normal to the frontal plane of the user may correspond to inhalation and exhalation of the user.

In some embodiments, a breath signal generation system may perform error determination steps before providing an estimated breath signal. For example, a breath signal generation system may generate a similarity score between an estimated breath signal for a user and a model breath signal. The similarity score may be then compared to a similarity threshold, where the estimated breath signal may be replaced by a default breath signal if the threshold fails to be satisfied. The default breath signal may be based on previous breath signals of a user and may be used to resemble an estimation of the user's current breath signal. The default breath signal may then be provided, if an actual breath signal deviates from the model breath signal by more than a threshold amount. A breath signal generation system may also determine, based on sensor data, whether error causing factors, for example, head motion or a steep head to body angle, are present. In such situations, the breath signal generation system may determine an error occurred during breath signal generation and inform the user that the error occurred and what factor may have caused the error.

In some embodiments, other biometric sensors, such as photo-plethysmography (PPG) sensors, may be integrated in a device; where data captured from these other sensors may be used alone or in combination with the image captured by the sensors of the device described above. For example, the data captured from such other sensors may be used to report current biometric data to the user as feedback, may be recorded for use in tracking biometric data over time, and so on.

In some embodiments, biometric data, including, but not limited to. respiration data, may be captured using at least some information from sensors on other devices external to the device, such as from a wristband. headphones, or earbuds.

### BRIEF DESCRIPTION OF THE DRAWINGS

FIG. 1A illustrates a region of interest encompassing an upper body of a user, according to some embodiments.

FIG. 1B illustrates a point cloud pertaining to landmarks of the upper body of the user, according to some embodiments.

FIG. 2 illustrates different possible estimated breath signals of a user, according to some embodiments.

FIG. 3 illustrates possible arrangements for image sensors, according to some embodiments.

FIG. 4 illustrates different portable devices with image sensors, according to some embodiments.

FIG. 5 illustrates different bodily poses a user may be performing, according to some embodiments.

FIG. 6 is a block diagram illustrating collection and processing of image data of a user to estimate a breath, according to some embodiments.

FIG. 7 is a flow chart of a method for capturing and processing point cloud data in a device to estimate a breath signal, according to some embodiments.

FIG. 8 is a flow chart of a method for capturing and processing point cloud data to estimate a breath signal in a device that may improve the signal-to-noise ratio, according to some embodiments.

FIG. 9 is a flow chart of a method for capturing and processing data from an image sensor to generate a point cloud which may include components and implemented methods as illustrated in FIGS. 3-5, according to some embodiments.

FIG. 10 illustrates an example head-mounted device (HMD) which collects and sends image data to an external device, receiving an estimated breath signal from the external device, according to some embodiments.

FIG. 11 is a block diagram illustrating an example device that may include components that may implement methods, as illustrated in FIGS. 1 through 9, according to some embodiments.

This specification includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

“Comprising.” This term is open-ended. As used in the claims, this term does not foreclose additional structure or steps. Consider a claim that recites: “An apparatus comprising one or more processor units . . . ” Such a claim does not foreclose the apparatus from including additional components (e.g., a network interface unit, graphics circuitry, etc.).

“Configured To.” Various units, circuits, or other components may be described or claimed as “configured to” perform a task or tasks. In such contexts, “configured to” is used to connote structure by indicating that the units/circuits/components include structure (e.g., circuitry) that performs those task or tasks during operation. As such, the unit/circuit/component can be said to be configured to perform the task even when the specified unit/circuit/component is not currently operational (e.g., is not on). The units/circuits/components used with the “configured to” language include hardware-for example, circuits, memory storing program instructions executable to implement the operation, etc. Reciting that a unit/circuit/component is “configured to” perform one or more tasks is expressly intended not to invoke 35 U.S.C. § 112, paragraph (f), for that unit/circuit/component. Additionally, “configured to” can include generic structure (e.g., generic circuitry) that is manipulated by software or firmware (e.g., an FPGA or a general-purpose processor executing software) to operate in manner that is capable of performing the task(s) at issue. “Configure to” may also include adapting a manufacturing process (e.g., a semiconductor fabrication facility) to fabricate devices (e.g., integrated circuits) that are adapted to implement or perform one or more tasks.

“First,” “Second,” etc. As used herein, these terms are used as labels for nouns that they precede, and do not imply any type of ordering (e.g., spatial, temporal, logical, etc.). For example, a buffer circuit may be described herein as performing write operations for “first” and “second” values. The terms “first” and “second” do not necessarily imply that the first value must be written before the second value.

“Based On” or “Dependent On.” As used herein, these terms are used to describe one or more factors that affect a determination. These terms do not foreclose additional factors that may affect a determination. That is, a determination may be solely based on those factors or based, at least in part, on those factors. Consider the phrase “determine A based on B.” While in this case, B is a factor that affects the determination of A, such a phrase does not foreclose the determination of A from also being based on C. In other instances, A may be determined based solely on B.

“Or.” When used in the claims, the term “or” is used as an inclusive or and not as an exclusive or. For example, the phrase “at least one of x, y, or z” means any one of x, y, and z, as well as any combination thereof.

### DETAILED DESCRIPTION

Various techniques estimating a breath signal of a user of a device using point cloud data are described. Point cloud data may store or otherwise represent points in three-dimensional space. Points may be associated with otherwise describe objects in the space. Point cloud data may be described using various types of coordinate systems in order to describe the positions or locations of the points in three-dimensional space. As objects in the space, such as a user, may move, the point cloud data may change over time to describe the movement of the object in space over time.

Point cloud data may provide robust information for measuring biometric information, in some scenarios. A breath signal, for instance, can be estimated according to various techniques discussed below because the evidence of the breath signal can be captured by point cloud data according to a user's body movement in a space. For example, point cloud data associated with an upper body of the user may be obtained and used to estimate the breath signal.

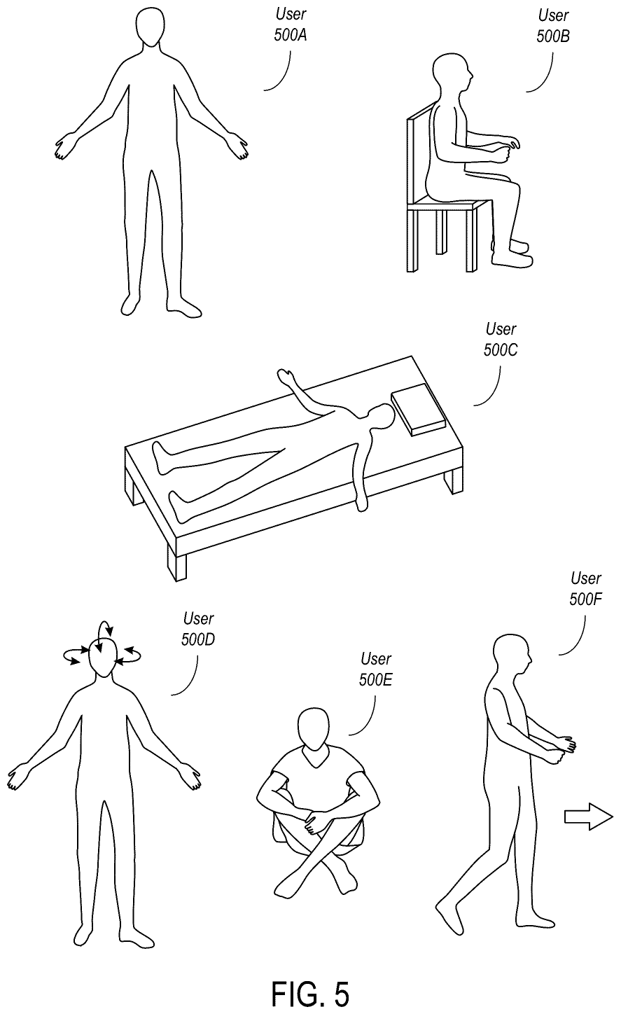

Because point cloud data is used to estimate a breath signal of a user, it can be appreciated that estimation techniques can adapt to a wide variety of body positions of the user. For example, as discussed in detail below with regard to FIG. 5, different body positions may involve different regions of interest that are captured according to the placement of sensors relative to the user. Point cloud data-based estimation techniques can accommodate these different positions and there expand the capabilities of breath estimation techniques to be implemented for a variety of different types of applications that may utilize the estimated breath signal. For instance, interactive applications that use the estimated breath signal based on user movements or interactions can handle a larger number of user movements instructed by or utilized by the application without sacrificing the ability to estimate a breath signal of the users. Accordingly, it may be apparent to one of skill in the art that using point cloud data to estimate breath techniques can improve the performance of breath signal estimation technologies and downstream systems that rely upon breath signal information.

Point cloud data can be obtained in different ways. In some embodiments, image data of the user may be captured using image sensors and analyzed to derive the point cloud data for a scene captured in the image data. In some embodiments, image sensors and/or depth sensors (e.g., stereo vision sensors and time-of-flight (ToF) sensors accordingly) may be located on a device with the sensor's field of view facing towards a user, for example, a cellphone or a head-mounted display (HMD), and data from those sensors may be used to determine a point cloud data. In some embodiments, image sensors may be implemented in a monocular or binocular configuration, and the sensors may include depth sensors used to determine point cloud data. In some embodiments, image sensors may be located on a different device and the image data may be transmitted via a wireless or wired connection. In some embodiments, a system may include multiple different devices which may communicate via wired or wireless connections in order to obtain point cloud data and utilize the point cloud data to estimate the user's breath signal.

In some embodiments, a generated or received point cloud data may be converted from local coordinates (e.g., image sensor coordinates) to world coordinates, and a measurement of movement of points of the point cloud may be performed in world coordinates. Converting the point cloud data from local coordinates to world coordinates may facilitate the estimation of a breath signal for a user when the user is moving around an environment, improving the accuracy of the estimated breath signal.

In some embodiments, other sensors such as depth sensors may be integrated in a device; data captured from these depth sensors may be used alone or in combination with data captured from image sensors to generate a point cloud and then, estimate a breath signal of a user based on the generated point cloud. For example, a time-of-flight depth sensor may be used alone to generate a point cloud encompassing an upper body of a user, or may be used in combination with stereo image sensors to improve accuracy of the point cloud.

In some embodiments, a received or generated point cloud data by a device may be used to identify points of the point cloud associated with landmark points of a body of a user, and the landmark points may be used to select a region of interest (ROI) encompassing at least part of an upper body of the user. Also, the breath signal generation system may use the ROI to determine which portions of the three-dimensional volumetric data are to be analyzed. In some embodiments, the ROI may be divided into two or more smaller subregions, and the smaller subregions may be utilized to measure multiple estimated subregion breath signals corresponding to each of the subregions. Also, each of the two or more subregion estimated breath signals may be combined into a single final breath signal according to a weighting method. For example, if there is a better quality of data for one sub-region as compared to the other sub-region (for example, due to a camera view angle), the breath signal for the sub-region with the better quality data may be weighted more heavily when determining a combined final breath signal. A breath signal generation system may minimize outlier data by dividing the ROI into smaller subregions, estimating subregion breath signals, and combining those subregion signals into a final breath signal according to a weighting method. Minimizing outlier data may improve the signal-to-noise ratio of the final breath signal. For example, if a user is wearing a scarf, measurements of depth of the scarf's surface relative to the frontal plane of the user over a period of time of the point cloud may generate an erroneous subregion breath signal. In some embodiments, outlier breath signals may be discarded, if deviating from breath signals for other subregions by more than a threshold amount. For example, when determining a combined final breath signal, an outlier sub-region breath signal may not be used in the calculation of the combined final breath signal. In other embodiments, outlier signals may be assigned a minimal weight such that the outlier signal does not meaningfully affect the combined final breath signal.

In some embodiments, the estimated breath signal may be compared to a model breath signal to generate a similarity score representative of the similarity between the signals. The model breath signal may be representative of an “ideal” signal or an accepted breath signal. In some embodiments, the similarity score may be compared to a similarity threshold. When the similarity threshold is not satisfied, the estimated breath signal may be replaced by a default breath signal, and when the threshold is satisfied, the estimated breath signal may be kept.

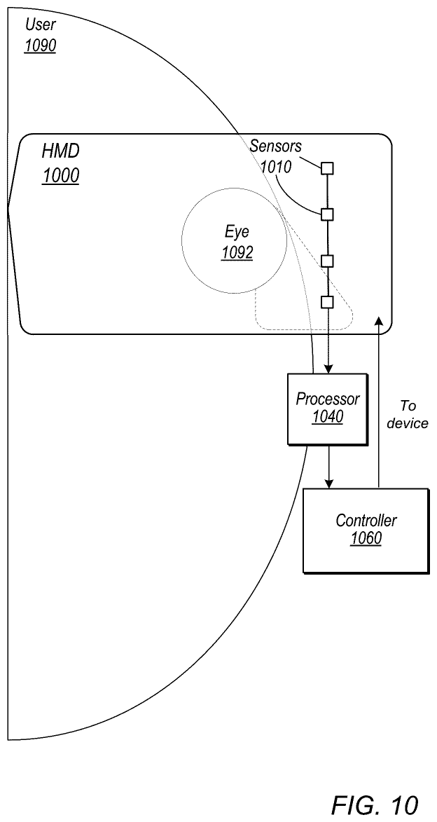

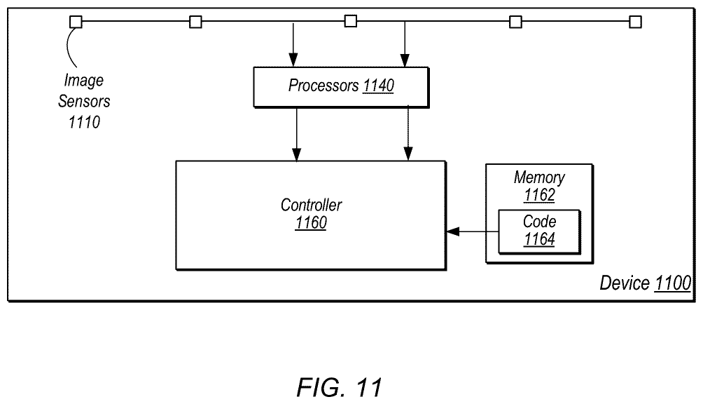

The methods and apparatus described herein may, for example, be implemented but not limited to a desktop device or in a portable device, such as a personal computer, a medical computer, a cellphone, a pad or tablet, etc. The methods and apparatus described herein may, as well, be implemented in a head-mounted device (HMD), such as a headset, helmet goggles, glasses, or other wearable device. Embodiments of example devices are further described with respect to FIGS. 10 and 11.

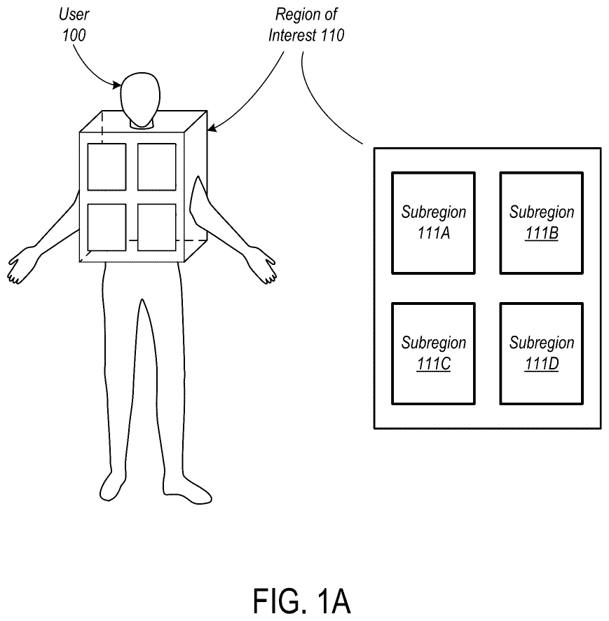

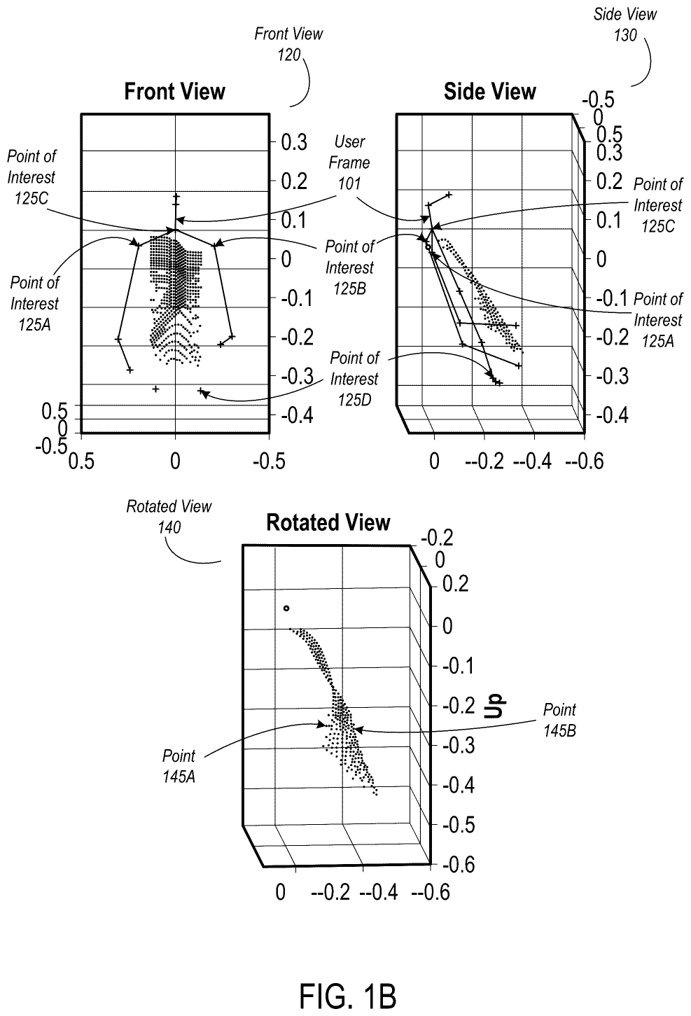

FIGS. 1A illustrates an example of a region of interest (ROI) 110 that may encompass an upper body of user 100. FIG. 1B illustrates a point cloud portrayed in different views with and without user frame 101. User frame 101 may be a representation of landmark features of a body of user 100.

As illustrated in FIG. 1A, the ROI 110 may be divided into 2 or more subregions. For example, FIG. 1A shows ROI 110 divided into 4 subregions, which include sub regions 111A, 111B, 111C and 111D.

FIG. 1B shows a point cloud seen in 3 different views: a front view 120, which shows the point cloud centered within user frame 101, a side view 130 and rotate view 140. As seen in the front view 120, the user frame may contain multiple points of interest, such as point of interest 125A, 125B, 125C and 125D associated with landmark points of the user frame 101. Note that the points of interest are also visible in side view 130 and the rotated view 140 which also shows points 145A and 145B, which may represent limits of an extent that a point of the point cloud may move over period of time.

In some embodiments, landmark points may be used to select the region of interest (ROI) 110. Landmark points may be selected based on a body size and shape of a user and may be used determine the ROI 110 for the user. A technique the breath signal generation system may use for selecting landmark points for determining the ROI 110 may be: (1) a first landmark point 125A may be selected from points of a point cloud, which may be to the right of a center of an upper body of a user (e.g. a right shoulder); (2) a second landmark point 125B may be selected to the left of the center (e.g. a left shoulder); (3) a first distance may be measured from the landmark points 125A to 125B; (4) a third landmark point 125C may be selected above the center of the upper body of the user (e.g. a connection between neck and shoulders); (5) a fourth landmark point 125D may be selected below the center (e.g. a point along a waist of the user); (6) a second distance may be measured from the landmark points 125C to 125D; (7) at least based on the first measured distance and the second measured distance, a width and a height may be calculated to form an area of interest; and (8) a depth may be selected such that when a ROI 110 is formed based on the area of interest and the depth, at least some of the points of the point cloud representation of the upper body may be encompassed within the ROI 110.

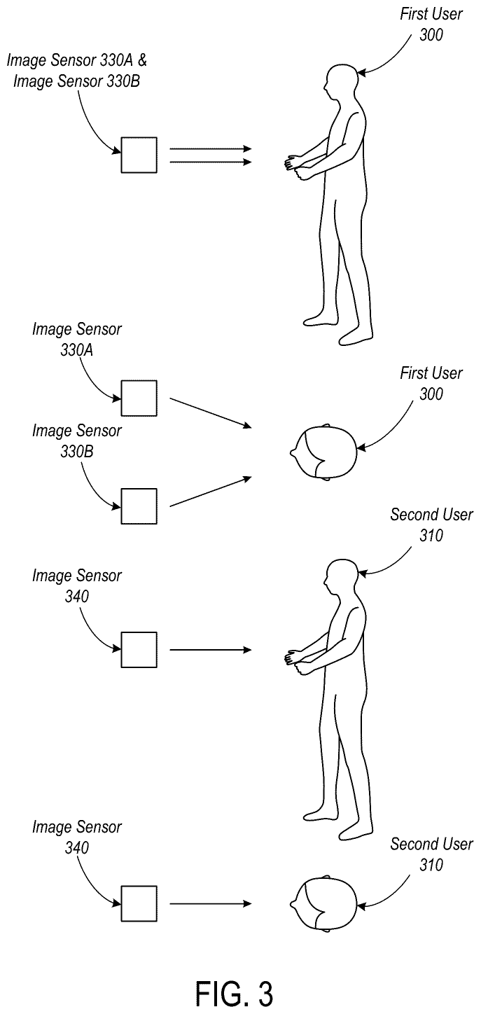

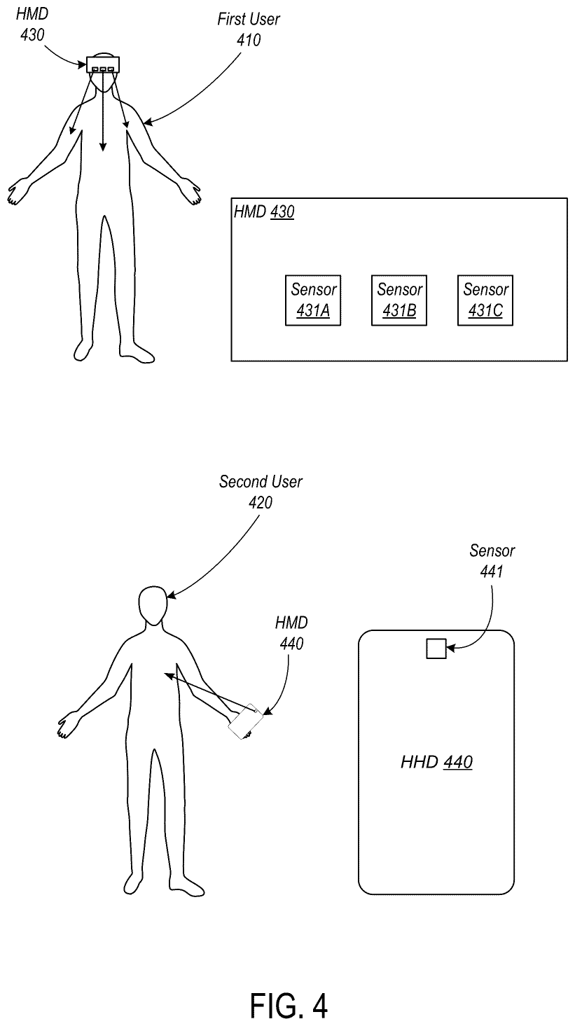

In some embodiments, the three-dimensional volumetric data, such as a point cloud representation, may be converted from a sensor coordinate system to a world coordinate system; and the selected ROI 110 may also be selected in the world coordinate system. Selecting the ROI 110 in the world coordinate system may allow for a dynamic ROI 110 that may move within the world coordinate system according to movements performed by the user 100. For example, when points come into and out of range of different cameras, the differences in coordinate systems between the cameras do not require reconfiguration if the points have already been transformed into a world coordinate system. Different possible arrangements for image sensors or other sensors and possible devices that may generate three-dimensional volumetric data, which may be used in the selection of the ROI 110 and in generating a breath signal, are illustrated in FIGS. 3 and 4.

In some embodiments, an estimated breath signal may be estimated based on a region of interest (ROI) 110 without dividing the ROI 110 into one or more subregions (e.g., sub-regions 111A-D). For example, all points of a point cloud over a period of time that are representative of a surface of an upper body of a user that is encompassed within a ROI 110 may be measured to estimate a single breath signal of the user.

In some embodiments, the points of the point cloud data associated with the upper body of the user that overlap with ROI 110, may move across some distances over a period of time. Points 145A and 145B may depict the limits of a distance traversed by a single first point when moving over the period of time (e.g., due to inhale and exhale). The measured point moving between point limits 145A and 145B alone, or in combination with other points, may be used to estimate a breath signal for the user.

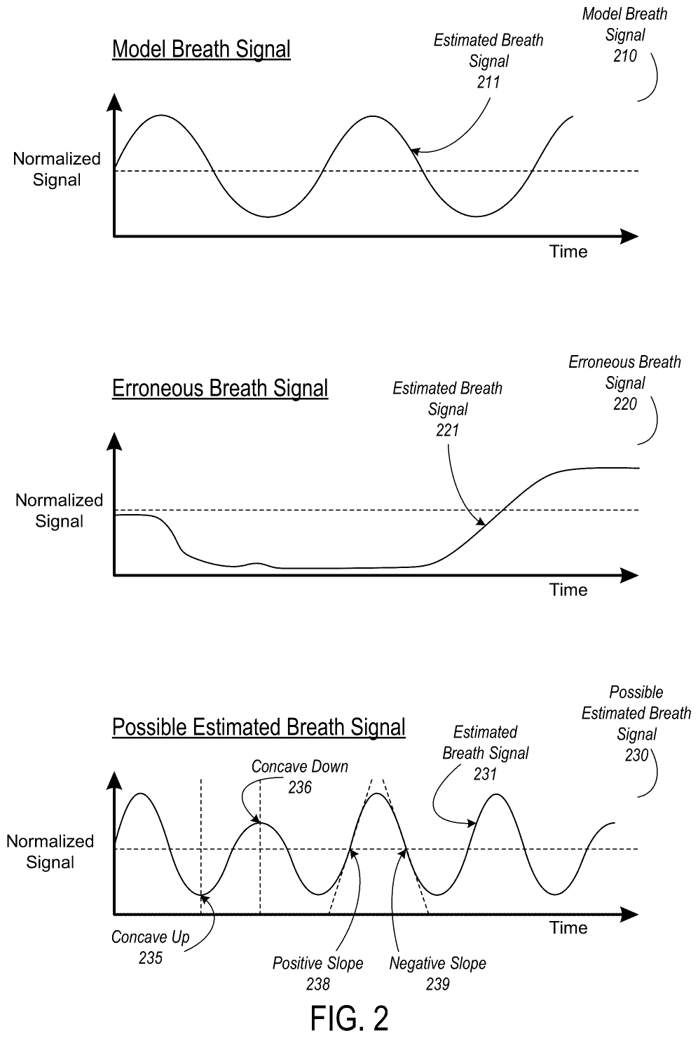

In some embodiments, the ROI 110 may be divided into smaller subregions such as subregions 111A, 111B, 111C and 111D, and the points within the ROI 110 may also be within one of the smaller subregions (e.g., points 145A and 145B may be within the subregion 111C). These points within each individual subregion may be measured over time, like point 145A and 145B, to estimate a breath signal for the user for each subregion of the ROI 110 (e.g., a first estimated breath signal for subregion 111A, a second estimated breath signal for subregion 111B, a third estimated breath signal for subregion 111C, and a fourth estimated breath signal for subregion 111D). These individual signals may then be combined according to a weighting method to generate a single estimated breath signal for the user that may have lower signal-to-noise ratio than an estimated breath signal if determined using only the ROI 110 (without division into subregions). For example, a breath signal produced using only the ROI 110 as a single region may include information from a subregion with a high signal-to-noise ratio. However, when using multiple sub-regions, the subregion might be weighted low as a result of the high signal-to-noise ratio, so a breath signal produced by combining subregion signals by a weighting method may include less of the noisy information from the subregion, and thus have a lower signal-to-noise ratio, than the breath signal produced using only the ROI 110 as a single region. Examples of estimated breath signals that may, for example, be calculated from a movement of a point along a distance from points 145A to 145B, are illustrated in FIG. 2.

While not shown in FIGS. 1A and 1B, in some embodiments, three-dimensional volumetric data associated with an upper body of a user may also include information associated with objects other than the upper body (e.g., arms and legs of the user, or a chair or a table close to the user). Prior to ROI identification, techniques may be implemented that may exclude such items from the three-dimensional volumetric data, such that the three-dimensional volumetric data includes less information related to objects other than the upper body. For example, points of the point cloud not within a sphere centered within the upper body with a radius of a selected distance (e.g., 1 meter) may be discarded from use in determining an estimated breath signal.

While not shown in FIGS. 1A and 1B, in some embodiments, the ROI 110 may be selected by observing three-dimensional volumetric data over a period of time, and selecting the ROI 110 based on points that may be oscillating, for example due to repeated expansion and contraction of the lungs of the user. The ROI 110 may be selected such that the ROI 110 may encompass at least a portion of the paths of the oscillating points.

While not shown in FIGS. 1A and 1B, in some embodiments, the ROI 110 may be selected by a neural network previously trained in identifying landmarks of the human body.

While not shown in FIGS. 1A and 1B, in some embodiments, three-dimensional volumetric data containing information associated with an upper body of a user may be generated from image data captured from one or more image sensors or depth data captured from one or more depth sensors, for example, time of flight LiDAR sensors.

FIG. 2 illustrates different possible estimated breath signals of a user, according to some embodiments.

Three breath signals are shown in FIG. 2: model breath signal 210, an erroneous breath signal 220, and a possible estimated breath signal 230. The model breath signal 210 may be a representation of an estimated breath signal 211 that completely represents a breath over time of the user. The erroneous breath signal 220 may be a representation of an estimated breath signal 221 that may not be depicting the users breath over time at all. The possible estimated breath signal 220 may be a representation of an estimated breath signal 231 that may be depicting fairly accurately the real breath of the user. The possible estimated breath signal 231 may contain none, one, or more of the following various components: (a) concave up 235, which may represent a user switching from inhale to exhale; (b) concave down 236, which may represent a user switching from exhale to inhale; (c) positive slope 238, which may represent a user during an inhale or exhale; (d) negative slope 239, which may represent a user during an exhale or inhale. In some embodiments, various values representative of a user's breath may be calculated from some or all of component's a-d. Although FIG. 2 shows 3 examples of possible estimations of breath signals of a user, the estimated breath signal may look like a combination of one or more of the examples signals, or may look differently.

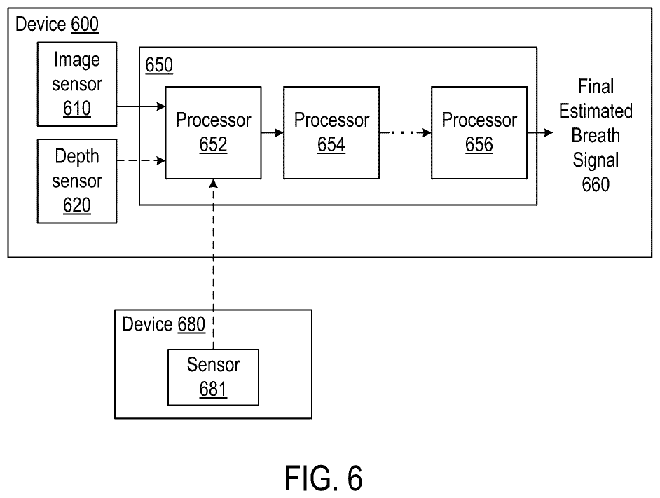

In some embodiments, model breath signal 211 may be computed from previous breath signals of a user, and the model breath signal 211 may be used as a benchmark for a comparison with a new estimated breath signal for the user. A similarity score may be generated from the comparison between model breath signal 211 and the new estimated breath signal, where the similarity score may represent how similar the waves may be. Then, the similarity score may be compared with a similarity threshold, where the similarity threshold may be the minimum required similarity between the estimated breath signal and the model breath signal 211. When the similarity threshold may be satisfied by the similarity score, the estimated breath signal may be provided to a destination. If the similarity score does not meet the similarity threshold, a default breath signal may replace the estimated breath signal, and the default breath signal may be provided. In some embodiments, the default breath signal may be calculated from previous estimated breath signals for the user. In some embodiments, the estimated breath signal may be stored within a memory (or other storage component) of a device for generating a default breath signal of the user. In some embodiments, the model breath signal 210 may be a pre-defined default signal stored within a memory of a device. An example of a system that may, for example, generate and analyze the estimated breath signal 230 in various embodiments is illustrated in FIG. 6.

While not shown in FIG. 2, in some embodiments, the possible estimated breath signal 230 may be segmented into two or more state segments corresponding with at least part of different stages of the breath cycle of the user: beginning of inhale, peak of inhale, ending of inhale, beginning of exhale, peak of exhale, and ending of exhale. The current state of a user may be provided to a destination with or without the breath signal.

In some embodiments, the model breath signal 210 may be a pre-defined signal stored within a memory of a device.

FIG. 3 illustrates possible arrangements for image sensors, according to some embodiments. FIG. 3 shows a first user 300 with image sensors 330A and 330B facing towards the user 300, and a second user 310 with image sensor 340 facing towards the user 310. Image sensors 330A and 330B may be a binocular configuration, and image sensor 340 may be a monocular configuration. This example has image sensors 330A-B and image sensors 340 facing a front body of the user 300 and user 310 respectively, and both users 300 and 310 are standing up. However, sensors 330A-B and sensor 340 may be located elsewhere, looking at a different view of user 300 and 310 respectively, and users 300 and 310 may be performing other body poses. In some embodiments, depth sensors may be used instead of image sensors.

In some embodiments, binocular sensors 330A and 330B may be stereo image sensors. In such embodiments, data captured from these stereo image sensors may be used alone or in combination with data from other image sensors to generate a point cloud containing points associated with an upper body of user 300. For example, a pair of stereo image sensors 330A-B may be used to generate a point cloud encompassing an upper body of user 300, pair of stereo image sensors 330A-B may be used in combination with other stereo image sensors or depth sensors to generate the point cloud or other three-dimensional volumetric data.

In some embodiments, monocular sensor 340 may include a depth sensor. In such embodiments, data captured from the depth sensor may be used alone or in combination with data from other image sensors to generate a point cloud containing points associated with an upper body of user 310. For example, a monocular depth sensor 340 may be used to generate a point cloud encompassing an upper body of user 310. The data data from the single depth sensor 340 may be used in combination with other depth sensors or stereo image sensors to generate the point cloud or other three-dimensional volumetric data.

Examples of head-mounted device (HMD) and a hand-held device that may, for example, include the binocular image sensor 330A-B and monocular image sensor 340 respectively are illustrated in FIG. 4.

FIG. 4 illustrates different portable devices with image sensors, according to some embodiments.

FIG. 4 illustrates two users: (1) a first user 410 wearing a head-mounted device (HMD) 430, where the HMD 430 includes sensors 431A, 431B, and 431C facing towards an upper body of user 430; and (2) a second user 420 holding a hand-held device (HHD) 440, where the HHD 440 includes sensor 441. Sensors 431A-C may be image sensors (e.g., visible light cameras and/or non-visible light cameras such as infrared and near infrared). This example shows HMD 430 with sensors 431A-C located towards the bottom of HMD 430, and HHD with sensor 441 located towards the top of HHD 440. However, sensors 431A-C may be located elsewhere on the HMD 430. Also, the HMD 430 may use a greater or smaller number of sensors. Also, sensor 441 may be located elsewhere on the HHD 440 and the HHD 440 may use more than one sensor 441.

In some embodiments, image sensors 431A-C may comprise of two or more stereo image cameras. Data collected from these stereo image cameras may be used to estimate point cloud data and a breath signal for a user.

In some embodiments, image sensor 441 may be used to estimate a breath signal for a user. In some embodiments, HHD 440 may contain other sensors not shown as a depth sensor, and data collected from the image sensor and the depth sensor may be used to estimate point cloud data and the breath signal.

In some embodiments, image sensors 431A-C and image sensor 441 may be depth sensors and may contain one or more image sensors as well. Data collected from these depth sensors, with or without data collected from the one or image sensors, may be used in generation of point cloud data and an estimation of a breath signal for a user.

Examples of different body poses that either user 410 or 420 may, for example, be performing while using the HMD 430 and HHD 440 are illustrated in FIG. 5.

FIG. 5 illustrates different body poses a user may be performing, according to some embodiments. FIG. 5 shows: (1) a user 500A standing upright; (2) a user 500B sitting on a chair; (3) a user 500C laying down; (4) a user 500D moving the user's 500D head; (5) a user 500E sitting down with the legs close to the user's 500E upper body; and (6) user 500F walking. The different body of user's 500A-F may be recorded by one or more image sensors to generate point cloud data of the respective user and a respective environment. In some embodiments, a single user may perform two or more of the poses of users 500A-B in any order, and the different poses of the single user may be captured by one or more image sensors to generate point cloud data. This example shows different body poses that a user may perform during a period of time while being recorded by one or more image sensors. However, there may be other body poses not shown in FIG. 5 that a user may perform while being recorded by the image sensors for which point cloud data may also be determined.

Examples of a head-mounted device (HMD) and a hand-held device (HHD) that may, for example, be worn by users 500A-F while performing each user's respective body pose are illustrated in FIG. 4.

While not shown in FIG. 5, in some embodiments, in addition to a body pose a user may be performing, other objects may be encountered to be within a close distance to and the other objects may be detected by the image sensor. This may result in three-dimensional volumetric data for a given object being generated. Methods to reduce the information included in three-dimensional volumetric data to information associated with an upper body of the user, or to discard information in three-dimensional volumetric data associated with objects other than the upper body of the user may be implemented.

FIG. 6 is a block diagram illustrating collection and processing of image data of a user to estimate a breath signal of the user in a device, according to some embodiments. A device 600 may include one or more image sensors 610. Sensors 610 may include stereo image sensors and/or monocular image sensors as described herein, for example, in FIGS. 3 and 4. The device 600 may also include one or more depth sensors 620 (e.g., time-of-flight LiDAR sensors or other depth sensors). The image sensors 610 and depth sensors 620 may communicate with a controller 650 of the device 600 that includes, but is not limited to, one or more processors and memory. The processors may include one or more processors 652 configured to pre-process signals from sensors 610 and/or sensors 620, one or more processors 654 configured to analyze the pre-processed signals to estimate a breath signal of a user. The one or more processors may also include one or more post-processors 656 configured to analyze the estimated breath signal prior to providing the final estimated breath signal 660.

Pre-processing signals from the sensors 610 and/or sensors 620 may include applying any of various signal processing techniques to generate a point cloud of a user. In some embodiments, pre-processing signals may include aligning the signals from two or more different sensors 610 and/or sensors 620. This may be necessary because signals from different sensors, or from different types of sensors, may not be temporally aligned. For example, a signal from an image sensor may show near-real-time correspondence with respiration, while a signal from a depth sensor may temporally lag behind actual respiration, as it may take varying amounts of time for signals from different sensors to arrive at processor 652. Thus, the signals from different types of sensors may need to be aligned before using them in combination to estimate a breath signal.

FIGS. 3 and 4 illustrate example configurations for different image sensors 610 and/or depth sensors 620 that may, for example, be integrated into device 600 in various embodiments.

Processors 654 may be configured to analyze and process data pre-processed by processor 652 to estimate a breath signal of a user. This breath signal may, for example, be provided to the user visually (graphically and/or textually) via a display of the device 600. In some embodiments, the breath signal may also be presented in audio for, for example via an audio signal to the user via headphones or earbuds. In some embodiments, the breath signal may be stored to memory of device 600. In some embodiments, the breath signal may be transmitted via a wired or wireless connection to an external device, such as a smartphone, pad or table, or laptop computer, and video or audio representations of the breath signal may be presented to the user via the external device, or stored on the external device. In some embodiments, the breath signal may be provided as an input to other applications which may use the breath signal to modify or generate content to be provided to a user of the device. For example, an image shown to the user may move in coordination with the breath signal.

In some embodiments, post-processing an estimated breath signal from processor 654 by processor 656 may include applying various signal processing techniques to the estimated breath signal. Pre-processing may include estimating the similarity of the estimated breath signal to a model breath signal as seen in FIG. 2 and providing a default signal if the estimated and model breath signals have no similitude. This may be necessary because signals from the different sensors 610 and/or 620 may collect more image data than simply the user. For example, the user for the estimated breath signal may be in close encounters with a second user for whom the estimated breath signal is not intended, and the breath data from the second user may be captured by image sensors 610 and/or 620. In such a case, the three-dimensional volumetric data of the second user may be used with the data from the intended first user, resulting in an erroneous estimated breath signal. Thus, the breath signal generation system may determine an error has occurred and provide a default breath signal to replace the erroneous estimated breath signal (e.g., model breath signal 2210 as discussed above with regard to FIG. 2, etc.). As another example, sensors 610 and/or 620 may have a field of view of an upper body of the user that is obstructed by clothing and may therefore estimate an erroneous breath signal. In such situations, the breath signal generation system may provide a default breath signal to replace the erroneous breath signal. The default breath signal may be computed from previous estimated breath signals of the user stored in a memory of device 600. The default breath signal may resemble a true breath signal of the user. In other embodiments, device 600 may lack previous estimated breath signals of the user stored in a memory of the device 600. In such embodiments, the device 600 may provide a default breath signal stored within memory of the device 600 to replace the erroneous estimated breath signal (e.g., a pre-set default breath model, etc.).

According to some embodiments, processing an estimated breath signal (e.g., by processors 652. 654, and 656, etc.) might include combining multiple captured signals (e.g., by image sensor(s) 610 and or depth sensor(s) 620, etc.), where each combined captured signal might be assigned a weight for the combination. For example, multiple signals may be captured which may include a field of view of an upper body of the user, where each respective captured signal, or the multiple captured signals, might comprise of a different captured portion of the upper body of the user. Each of the captured signals of the field of view of the upper body of the user may be similar to regions of interest (e.g., being captured by sensor(s) 610 and 620, etc.), as discussed above with regard to FIG. 1. Each of the multiple captured signals may have a weight assigned to each respective signal, and combining the multiple captured signals may generate the estimated breath signal for the user (e.g., which may then be comparted with a similarity score as discussed above with regard to FIG. 2, etc.).

In some embodiments, weights may be assigned to respective captured signals, from multiple captured signals, to generate the estimated breath signal for the user, based on confidence values provided by a trained neural network and additional factors. For example. a subregion of interest that includes few points may have a lower weight than a subregion of interest with more points. Properties of the signals may also be factors that influence weight, such as frequency-domain properties, which may enable the device 600 (e.g., processor(s) 652, 654 and/or 656, etc.) to objectively analyze the quality of the signals. For example, the device 600 may be able to determine a signal's consistency based on the frequency-domain. Consistency may be a frequency-domain property of a signal. A signal with a higher consistency may have a higher weight than a signal with a lower consistency. A signal to noise ration may be another property of a signal. A signal with a higher signal to noise ratio may have a higher weight than a signal with a low signal to noise ratio. the device 600 may determine a particular signal is not related to a true breath signal, and may remove the signal from further processing by assigning the signal no weight.

In other embodiments, the processors of device 600 (e.g., 652, 654 and/or 656, etc.), may analyze the generated estimated breath signal for the user, and determine the generated estimated breath signal is erroneous. The device 600 may analyze individual of the captured signals, as well as potential sources of error such as head motion and head to body angle. The device 600 may determine an uncertainty estimation, whether the signal is likely to be erroneous (e.g., similar to the similarity score discussed above with regard to FIG. 2, etc.), and what type of error likely occurred. The type of error which likely occurred may be classified based on the cause of the error, for example, and error caused by head motion may be a head motion error. The device 600 may provide an alternative default breath signal in place of an erroneous signal, as discussed above with regard to FIG. 2.

Although not illustrated, device 600 may include one or more inertial movement sensors, which may be utilized by device 600 (e.g., processors 652, 654 and/or 656, etc.) to estimate error of a generated breath signal for a user, according to some embodiments. A higher uncertainty estimation may be based on a high amount of head motion, which may be indicated by inertial data captured by the one or more inertial movement sensors, which may indicate that an error has occurred.

While not shown in FIG. 6, in some embodiments, other components of device 600 may be used in estimating a breath signal. In some embodiments, for example, visible light and/or IR sensors that are used for other purposes may collect data that is relevant to determining breath data. As an example, visible light may be used when capturing image data from the image sensor 610 for a better resolution. As another example, data from IR sensors may be pre-processed by processor 652 in conjunction with the image data from sensors 610 and/or sensors 620.

In some embodiments, breath data, including one or more of, but not limited to, image data, point cloud data, and breath signal data, may be captured by sensors 681 on one or more devices 680 external to device 600, for example from a security camera, a pad or tablet with one or more image sensors, or a desktop with one or more image sensors. Data captured from these sensors 681 in device(s) 680 may be pre-processed 652, analyzed 654, and may be post-processed 656, and may be used alone or in combination with the data captured by sensors 610 and/or sensors 620, for example to estimate the breath signal from different views, to report other variables related to breath as feedback, to be recorded for use in tracking the breath signal over time, and so on.

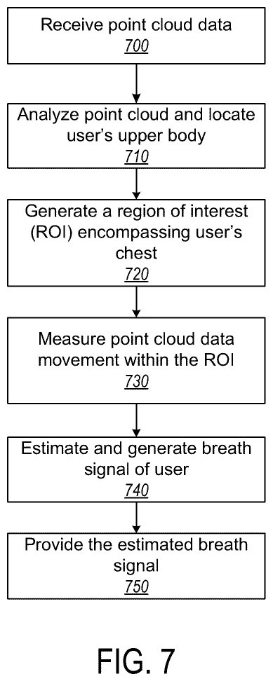

FIG. 7 is a flow chart of a method for capturing and processing point cloud data to estimate a breath signal of a user in a device, according to some embodiments.

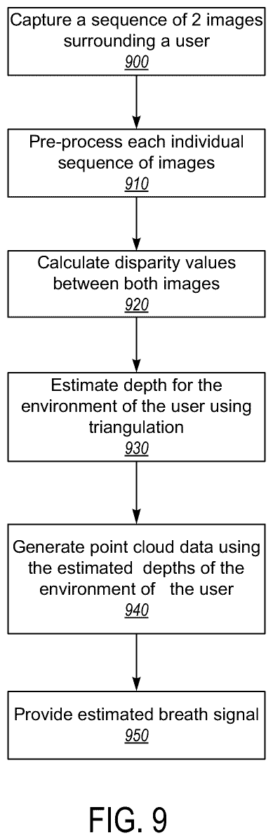

As indicated at 700, point cloud data may be received over wired or wireless connection containing data points associated with an upper body of a user and an environment of the user. As discussed below with regard to FIG. 9, the point cloud data may be generated from other captured sensor data (e.g., image data and/or depth information).

As indicated at 710, a breath signal generation system may analyze the point cloud data to identify points associated with the upper body. For example, a machine learning model may be trained to identify a region of interest based on upper body landmarks from point cloud data.

As indicated at 720, a breath signal generation system may generate a region of interest (ROI) based on the identified data points associated with the upper body, where the ROI may encompass the data associated with the upper body.

As indicated at 730, a breath signal generation system may measure the movement of data points of the point cloud data within the selected ROI.

As indicated at 740, a breath signal generation system may estimate a breath signal based on the measured movements of the data points within the ROI.

As indicated at 750, a breath signal generation system may provide the estimated breath signal. As an example, the breath signal may be provided to the user in visual and/or audio form or may be provided indirectly such as via modification to outputs of one or more other applications that receive the breath signal. The estimated breath signal may also be recorded for tracking breath signal data over time. Additionally, the estimated breath signal may be transmitted to an external device.

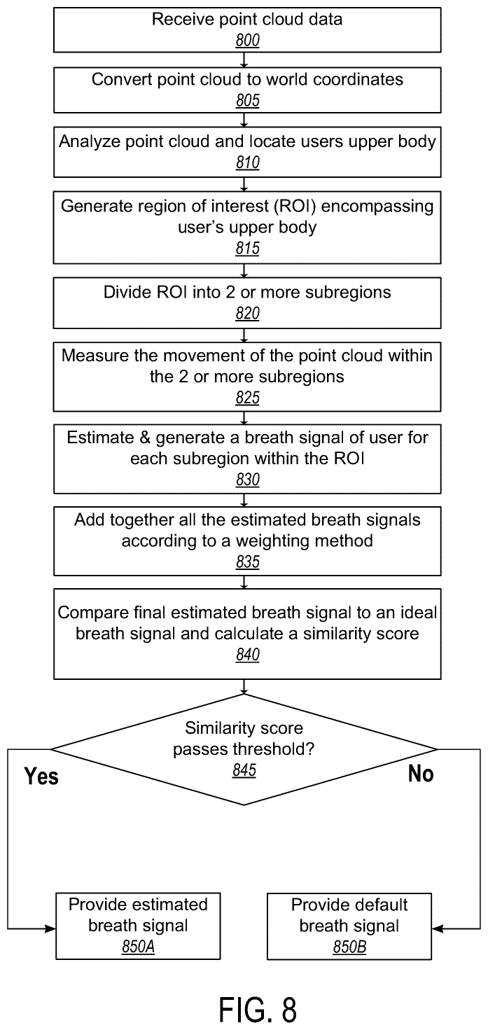

FIG. 8 is a flow chart of a method for capturing and processing point cloud data to estimate a breath signal in a device with a reduced signal-to-noise ratio, according to some embodiments.

As indicated at 800, a breath signal generation system may receive point cloud data, or other three-dimensional volumetric data, containing data points associated with an upper body of a user and an environment of the user.

As indicated at 805, a breath signal generation system may convert the received point cloud data from sensor coordinates to world coordinates.

As indicated at 810, a breath signal generation system may analyze the point

cloud in world coordinates to identify data points of the point cloud associated with the upper body of the user.

As indicated at 815, a breath signal generation system may generate a region of interest (ROI) based on the identified data points associated with the upper body, where the ROI may include the data points associated with the upper body.

As indicated at 820, a breath signal generation system may divide the selected ROI into two or more subregions, where the two or more subregions collectively form the whole ROI.

As indicated at 825, a breath signal generation system may measure the movement of the points of the point cloud within the two or more subregions independently.

As indicated at 830, a breath signal generation system may estimate and generate two or more independent breath signals for each of the two or more subregions based on the independent measurements of the points contained within each of the respective two or more subregions. As discussed above with regard to FIG. 6, the two or more independent breath signals may have weights assigned, where the assigned may be based on accuracy of each individual signal (e.g., subregions of interest containing few points may have a lower weight than a subregion of interest with more points, frequency-domain properties may affect assigned weights, etc.).

As indicated at 835, a breath signal generation system may combine the two or more independent estimated breath signals together according to a weighting technique to generate a final estimated breath signal. For example, a weighting technique that equally weights the two or more breath signals may be used. As another example, a weighting technique that weights the breath signals differently (e.g., according to confidence scores or other values associated with each breath signal or the source of sensor data used to estimate each breath signal) may be used. In some embodiments, a weighting technique may be selected to increase a signal-to-noise ratio. For example, a weighting technique may be selected where each estimated subregion breath signal is combined together using a weight according to how many data points are associated with each estimated subregion breath signal. As another example, the weighting method may combine subregion breath signals that are similar to each other, and discard subregion breath signals that are not similar to other subregion breath signals (e.g., signals which may be associated with lower weights, or signals which may be associated with zero weight, as discussed above with regard to FIG. 6, etc.).

As indicated at 840, a breath signal generation system may compare the final estimated breath signal to a model breath signal, and calculate a similarity score between the estimated and model signals.

As indicated at 845, a breath signal generation system may analyze the similarity score to determine if the similarity score satisfies a similarity threshold.

As indicated at 850A, if the similarity score satisfies the threshold, the final estimated breath signal is provided.

As indicated at 850B, if the similarity score fails to satisfy the threshold, a default breath signal is provided. The final estimated breath signal or the default breath signal may be provided, for example, to the user in visual and/or audio form (or indirectly via one or more applications). The estimated breath signal may also be recorded for use in tracking breath signal data over time. Also, the estimated breath signal may be transmitted to an external device via a wired or wireless connection.

In some embodiments, a default breath signal may be computed from previous estimated breath signals for a user, where the previous estimated breath signals are stored in memory of a device. For example, a particular default breath signal may be computed for a particular user such that the particular default breath signal may be similar to the true breath signal of the user.

FIG. 9 is a flow chart of a method for capturing and processing data from an image sensor to generate a point cloud in a device, according to some embodiments. As indicated at 900, a breath signal generation system may capture data of two image data streams comprising at least an upper body of a user using stereo image sensors.

As indicated at 910, a breath signal generation system may pre-process the raw image data captured by the two stereo image sensors, for example to align signals from different sensors that are out of phase.

As indicated at 920, a breath signal generation system may analyze the pre-processed sequences of image data to calculate disparity values between the first and second stream of image data.

As indicated at 930, a breath signal generation system may use the calculated disparity values to estimate respective depths of the elements and objects contained within the streams of image data. The breath signal generation system may use a triangulation method to estimate such depths.

As indicated in 940, a breath signal generation system may use the estimated depths and the sequences of image data to generate a point cloud containing points that may be representative of landmarks of the upper body of the user and points that may be representative of an environment of the user. FIG. 1B illustrates an example of a point cloud containing points associated with an upper body of a user which may, for example, be at least part of the point cloud generated in 940.

As indicated at 950, a breath signal generation system may provide the generated point cloud data. For example, such point cloud data may be transmitted to an external device to be used to generate a breath signal. The generated point cloud may also be further computed within the first device, or may be presented to the user in some visual form.

The following sections further describe image-based respiration detection methods and apparatus using image sensors integrated in, or attached to, a device as described in reference to FIGS. 1 through 9.

Respiration detection can be performed using a band worn by a subject and/or using a flow-meter in-line with the subject's mouth or nose (or both). These techniques, however, are not designed for long-term user comfort, and are not easily integrated with many devices. As discussed above, as well as in further detail below, image sensors may be implemented to integrate with signal processing and processors of a device. These image sensors may be adapted for use in image-based breath signal estimation as described herein. Utilizing the image sensors to estimate breath signal in a device as described herein may provide a more convenient measure of breath signal for a user.

Embodiments of an unobtrusive, non-contact method for breath signal estimation that uses three-dimensional volumetric data are described. Embodiments are described that use one or more image sensors that include a pair of stereo cameras; the stereo cameras are arranged such that the movement caused by normal respiration are measured. Some embodiments may leverage a multi-sensor array of image sensors (depth sensors and/or stereo image sensors) which can provide more detail, and the system may be configured to adaptively sub-sample only the necessary pixels from the array of image data that is used for breath signal estimation.

Detecting breath signals may be used in many applications. Breath signals may, for example, be used as a non-invasive method of tracking a user's physiological and emotional state. For example, respiration data may indicate a fight-or-flight response (stress, anxiety). In such a response, respiration may increase. Sensors as described herein may detect motion of the user's chest, and this motion may be analyzed to estimate a breath signal from which changes in a respiration pattern may be detected. The breath signal may be used to distinguish between different affective states (e.g., stress vs embarrassment).

The generated breath signal may be presented to the user using visual, audio, or other methods. This information may, for example, be used in an application to enhance relaxation via biofeedback. The breath signal may also be recorded. The recorded data may, for example, be used to track biometric data over time. Biometric data captured on the device may also be transmitted to another device such as a smartphone, tablet, or notebook computer and displayed or stored on that device.

Security measures such as encryption and/or password protection may be implemented in software and/or hardware on the device to ensure that biometric data for a user generated or stored on a device using the image sensors as described herein is protected and kept safe. For example, when biometric data is transmitted, the biometric data may be encrypted and only accessible by other applications or devices after being granted permission to access the biometric data by the user.

In some embodiments, the system may be configured to additionally use data from wearable sensors (e.g. a PPG sensor on a watch or wristband) and/or other sensors to increase breath signal accuracy, contextual awareness, and to reduce time to first breath signal output.

In some embodiments, the system may be configured to provide visual and/or audio feedback to the user utilizing the device based on the breath signal captured by the image sensors and processed by the processors. For example, respiration sounds may be generated and fed to the user via earbuds or headphones based on the breath signal determined from the image data captured by the image sensors integrated in the device. This may, for example provide the user with a richer experience, or more immersive experience, while utilizing an application implemented on the device.

Some embodiments of a device may thus include one or more integrated image sensors configured to detect the chest region of a user. One or more of the image sensors may be connected to a processor and a signal processing chain capable of adaptively estimating breath signal. In some embodiments, the processor and signal processing chain may leverage data from additional sensors of the device such as depth sensors to process signals from the image sensors. Some embodiments may include a frequency domain signal processing system based on lock-in amplification techniques configured to accept input from additional sensors to exclude non-informative motion relative to the user from breath signal generation. In some embodiments, the breath signal generation system may be configured to track long term chest movements and generate a continual breath signal as part of a suite of respiration and health quantifying applications of the device and/or external devices. In some embodiments, a breath signal generation system may use data from other wearable sensors (e.g. PPG sensors on a watch or wristband) to increase breath signal accuracy and to reduce time to initial breath signal output.

In some embodiments, data from other sensors including but not limited to inertial sensors, inertial measurement units (IMU) measuring head and body movement, image or depth sensors directed at other portions of the body such as the chest or back, data from one or more microphones that capture breath sounds, and motion sensors that detect motion of the diaphragm, shoulders, or other body parts during breathing may be captured and analyzed along with the image and depth sensor data to determine and track respiration rate.

Embodiments of methods and apparatus for estimating a breath signal for a user as described herein may, for example, be used in head-mounted displays (HMD), for example HMDs of computer-generated reality (XR) systems such as a mixed or augmented reality (MR) system or virtual reality (VR) systems.

A device that implements methods and apparatus for estimating a breath signal for a user as illustrated in FIGS. 1A through 9 may include a controller comprising one or more processors and memory. Controller may include one or more of various types of processors, image signal processors (ISPs), graphics processing units (GPUs), coder/decoders (codecs), and/or other components for processing and rendering video and/or images. In some embodiments, at least some of the functionality of the controller may be implemented by an external device coupled to the device by a wired or wireless connection. In some embodiments, the controller may be coupled to an external memory for storing and reading data and/or software.

FIG. 10 illustrates an example head-mounted device (HMD) that may include components and implemented methods as illustrated in FIGS. 1 through 9, according to some embodiments. An HMD 1000 may, for example, be a component in a mixed or augmented reality (MR) system. Note that HMD 1000 as illustrated in FIG. 10 is given by way of example, and is not intended to be limiting. In various embodiments, the shape, size, and other features of an HMD 1000 may differ, and the locations, numbers, types, and other features of the components of an HMD 1000 may vary. In some embodiments, HMD 1000 may include, but is not limited to, a display and two optical lenses (eyepieces) (not shown), mounted in a wearable housing or frame. Alternatively, HMD 1000 may include a display but not eyepieces. As shown in FIG. 10, HMD 1000 may be positioned on the user's head 1090 such that the display is disposed in front of the user's eyes 1090. The HMD 1000 may also include one or image sensors 1010 as described herein in reference to FIGS. 1A through 9.

A controller 1060 for the MR system may be implemented in the HMD 1000, or alternatively may be implemented at least in part by an external device (e.g., a computing system) that is communicatively coupled to HMD 1000 via a wired or wireless interface. Controller 1060 may include one or more of various types of processors, image signal processors (ISPs), graphics processing units (GPUs), coder/decoders (codecs), and/or other components for processing and rendering video and/or images. Controller 1060 may render frames (each frame including a left and right image) that include virtual content based at least in part on inputs obtained from the sensors, and may provide the frames to the display.

The HMD 1000 may include one or more processors 1040 configured to pre-process signals from the sensors 1010 as described herein; controller 1060 may be configured to analyze the pre-processed signals to estimate, generate, and output breath information including but not limited to breath signal. The breath information may be output to the display. Instead or in addition, breath information may be provided in audible form to the user, for example via earbuds or headphones coupled to or integrated in the HMD 1000. In some embodiments, breath information may be recorded, for example to memory of the HMD; the recorded breath signal data may, for example, be used to track changes in respiration over time. In some embodiments, breath information may be transmitted to another device via a wired or wireless connection.

Embodiments of an HMD 1000 as illustrated in FIG. 10 may, for example, be used in augmented or mixed (AR) applications to provide augmented or mixed reality views to the user 1090. HMD 1000 may include one or more sensors, for example located on external surfaces of the HMD 1000, which collect information about the user 1090′s external environment (video, depth information, lighting information, etc.); the sensors may provide the captured information to controller 1060 of the MR system. The sensors may include one or more stereo vision cameras that capture a sequence of images of the user's environment that may be used to provide the user 1090 with a virtual view of their real environment. In some embodiments, video streams of the real environment captured by the visible light cameras may be processed by the controller 1060 of the HMD 1000 to render augmented or mixed reality frames that include virtual content overlaid on the view of the real environment, and the rendered frames may be provided to the HMD 1000′s display system.

FIG. 11 is a block diagram illustrating an example device 1100 that may include components and implemented methods as illustrated in FIGS. 1 through 9, according to some embodiments. In some embodiments, the device 1100 may implement any of various types of display technologies. For example, the device 1100 may include one or more display systems that displays frames that are viewed by a user. The one or more display system may, for example, be a DLP (digital light processing), LCD (liquid crystal display), or LCOS (liquid crystal on silicon) technology display system. Note that other types of displays may be used in some embodiments.

In some embodiments, device 1100 may include a controller 1160 configured to implement functionality of the device and to generate frames that are provided to the device's one or more displays. In some embodiments, device 1100 may also include a memory 1162 configured to store software (code 1164) of the device that is executable by the controller 1160. In some embodiments, device 1100 may also include one or more interfaces (e.g., a Bluetooth technology interface, USB interface, etc.) configured to communicate with an external device via a wired or wireless connection. In some embodiments, at least a part of the functionality described for the controller 1160 may be implemented by an external device. The external device may be or may include any type of computing system or computing device, such as a desktop computer, notebook or laptop computer, pad or tablet device, smartphone, hand-help computing device, game controller, game system, medical system and so on.

In various embodiments, controller 1160 may be a uniprocessor system including one processor, or a multiprocessor system including several processors (e.g., two, four, eight, or another suitable number). Controller 1160 may include central processing units (CPUs) configured to implement any suitable instruction set architecture, and may be configured to execute instructions defined in that instruction set architecture. For example, in various embodiments controller 1160 may include general-purpose or embedded processors implementing any of a variety of instruction set architectures (ISAs), such as the x86, PowerPC, SPARC, RISC, or MIPS ISAs, or any other suitable ISA. In multiprocessor systems, each of the processors may commonly, but not necessarily, implement the same ISA. In some embodiments, controller 1160 may be implemented as a system on a chip (SoC). For example, in some embodiments, processors, memory, I/O interface (e.g. a fabric), etc. may be implemented in a single SoC comprising multiple components integrated into a single chip. For example an SoC may include multiple CPU cores, a multi-core GPU, a multi-core neural engine, cache, one or more memories, etc. integrated into a single chip. In some embodiments, an SoC embodiment may implement a reduced instruction set computing (RISC) architecture, or any other suitable architecture. Controller 1160 may employ any microarchitecture, including scalar, superscalar, pipelined, superpipelined, out of order, in order, speculative, non-speculative, etc., or combinations thereof. Controller 1160 may include circuitry to implement microcoding techniques. Controller 1160 may include one or more processing cores each configured to execute instructions. Controller 1160 may include one or more levels of caches, which may employ any size and any configuration (set associative, direct mapped, etc.). In some embodiments, controller 1160 may include at least one graphics processing unit (GPU), which may include any suitable graphics processing circuitry. Generally, a GPU may be configured to render objects to be displayed into a frame buffer (e.g., one that includes pixel data for an entire frame). A GPU may include one or more graphics processors that may execute graphics software to perform a part or all of the graphics operation, or hardware acceleration of certain graphics operations. In some embodiments, controller 1160 may include one or more other components for processing and rendering video and/or images, for example image signal processors (ISPs), coder/decoders (codecs), etc.

Memory 1162 may include any type of memory, such as dynamic random access memory (DRAM), synchronous DRAM (SDRAM), double data rate (DDR, DDR2, DDR3, etc.) SDRAM (including mobile versions of the SDRAMs such as mDDR3, etc., or low power versions of the SDRAMs such as LPDDR2, etc.), RAMBUS DRAM (RDRAM), static RAM (SRAM), etc. In some embodiments, one or more memory devices may be coupled onto a circuit board to form memory modules such as single inline memory modules (SIMMs), dual inline memory modules (DIMMs), etc. Alternatively, the devices may be mounted with an integrated circuit implementing system in a chip-on-chip configuration, a package-on-package configuration, or a multi-chip module configuration.

In some embodiments, the device 1100 may include one or more sensors that collect information about the user's environment (video, depth information, lighting information, etc.). The sensors may provide the information to the controller 1160 of the device. In some embodiments, the sensors may include, but are not limited to, visible light cameras (e.g., video cameras) and ambient light sensors.

The device 1100 may also include one or more image sensor 1110 as described herein in reference to FIGS. 1A through 9. The device 1100 may include one or more processors 1140 configured to pre-process signals from the sensors 1110 as described herein; controller 1160 may be configured to analyze the pre-processed signals to estimate, generate, and output breath information including but not limited to breath rate information. The breath rate information may be output to the display. Instead or in addition, breath rate information may be provided in audible form to the user, for example, via earbuds or headphones coupled to or integrated in the device 1100. In some embodiments, breath information may be recorded, for example to memory 1162; the recorded breath data may, for example, be used to track changes in respiration over time. In some embodiments, breath information may be transmitted to another device via a wired or wireless connection.

A real environment refers to an environment that a person can perceive (e.g. see, hear, feel) without use of a device. For example, an office environment may include furniture such as desks, chairs, and filing cabinets; structural items such as doors, windows, and walls; and objects such as electronic devices, books, and writing instruments. A person in a real environment can perceive the various aspects of the environment, and may be able to interact with objects in the environment.

An extended reality (XR) environment, on the other hand, is partially or entirely simulated using an electronic device. In an XR environment, for example, a user may see or hear computer generated content that partially or wholly replaces the user's perception of the real environment. Additionally, a user can interact with an XR environment. For example, the user's movements can be tracked and virtual objects in the XR environment can change in response to the user's movements. As a further example, a device presenting an XR environment to a user may determine that a user is moving their hand toward the virtual position of a virtual object, and may move the virtual object in response. Additionally, a user's head position and/or eye gaze can be tracked and virtual objects can move to stay in the user's line of sight.

Examples of XR include augmented reality (AR), virtual reality (VR) and mixed reality (MR). XR can be considered along a spectrum of realities, where VR, on one end, completely immerses the user, replacing the real environment with virtual content, and on the other end, the user experiences the real environment unaided by a device. In between are AR and MR, which mix virtual content with the real environment.

VR generally refers to a type of XR that completely immerses a user and replaces the user's real environment. For example, VR can be presented to a user using a head mounted device (HMD), which can include a near-eye display to present a virtual visual environment to the user and headphones to present a virtual audible environment. In a VR environment, the movement of the user can be tracked and cause the user's view of the environment to change. For example, a user wearing a HMD can walk in the real environment and the user will appear to be walking through the virtual environment they are experiencing. Additionally, the user may be represented by an avatar in the virtual environment, and the user's movements can be tracked by the HMD using various sensors to animate the user's avatar.

AR and MR refer to a type of XR that includes some mixture of the real environment and virtual content. For example, a user may hold a tablet that includes a camera that captures images of the user's real environment. The tablet may have a display that displays the images of the real environment mixed with images of virtual objects. AR or MR can also be presented to a user through an HMD. An HMD can have an opaque display, or can use a see-through display, which allows the user to see the real environment through the display, while displaying virtual content overlaid on the real environment.

There are many types of devices that allow a user to experience the various forms of XR. Examples include HMDs, heads up displays (HUDs), projector-based systems, smart windows, tablets, desktop or laptop computers, smart watches, earbuds/headphones, controllers that may include haptic devices, and many others. As mentioned above, an HMD, or any of the other devices listed above may include opaque displays (e.g. liquid crystal displays (LCDs), organic light emitting diode (OLED) displays or micro-LED displays) or see through displays. A see through display can have a medium through which light is directed to a user's eyes. The medium can include one or more of a waveguide, hologram medium, optical combiner, optical reflector and other optical components. An image can be generated and propagated through the medium using a display source such as OLEDs, micro-LEDs, liquid crystal on silicon (LCOS), a light scanner, digital light projection (DLP).

Devices for XR may also include audio output devices such as speakers to present audio (including spatial audio) to users, haptics devices to stimulate the user's sense of touch, and other devices to stimulate any of the user's senses. Additionally, the device may include numerous sensors, including cameras, microphones, depth sensors. eye tracking sensors, environmental sensors, input sensors, and other sensors to allow the device to understand the user and the real environment.

The methods described herein may be implemented in software, hardware, or a combination thereof, in different embodiments. In addition, the order of the blocks of the methods may be changed, and various elements may be added, reordered, combined, omitted, modified, etc. Various modifications and changes may be made as would be obvious to a person skilled in the art having the benefit of this disclosure. The various embodiments described herein are meant to be illustrative and not limiting. Many variations, modifications, additions, and improvements are possible. Accordingly, plural instances may be provided for components described herein as a single instance. Boundaries between various components, operations and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

What is claimed is:

1. A system, comprising:
one or more image sensors configured to collect image data of a user in an environment over a period of time; and
processing circuitry, configured to:
obtain the image data;
generate, from the image data, point cloud data comprising points representative of the user in the environment as captured in the image data over the period of time;
select one or more regions of interest in the point cloud data across the period of time, wherein the selected one or more regions of interest contain points in the point cloud data that correspond to upper-body landmarks of the user;
measure respective motion of the points contained in the selected one or more regions of interest in the point cloud data over the period of time;
based on the respective motion of the points, generate an estimated breath signal of the user; and
provide a final breath signal based, at least in part, on the estimated breath signal.

2. The system as recited in claim 1,
wherein to measure the respective motion of the points contained in the selected one or more regions of interest in the point cloud data over the period of time, the processing circuitry is configured to:
divide the one or more regions of interest into two or more smaller subregions;
independently measure the respective motion of the points contained in the two or more subregions over the period of time;
wherein to generate an estimated breath signal of the user, the processing circuitry is configured to:
based on the respective motion of the points in each of the two or more subregions, generate respective subregion breath signals; and
combine the subregion breath signals according to a weighting method to generate the estimated breath signal.

3. The system as recited in claim 1, wherein to select the region of interest, the processing circuitry is configured to:
select a first reference point and a second reference point from the point cloud data corresponding to respective ones of the upper-body landmarks of the user as an upper boundary and lower boundary;
measure a first distance from the first reference point to the second reference point;
select a third reference point and a fourth reference point corresponding to different ones of the upper-body landmarks of the user as a left boundary and a right boundary;
measure a second distance from the third reference point to the fourth reference point;
determine height and width for the region of interest to create an area of interest based on the first distance and the second distance;
select a depth for the area of interest to create the region of interest, wherein the depth is selected such that when the region of interest is centered between the first and second reference points and the third and fourth reference points at least part of the points in the point cloud data corresponding to the upper-body landmarks of the user are contained within the region of interest.

4. The system as recited in claim 1, wherein to provide the final breath signal, the processing circuitry is configured to:
compare the estimated breath signal to a model breath signal to generate a similarity score between the estimated breath signal and the model breath signal; and
compare the similarity score to a similarity threshold, where the processing circuitry is configured to:
provide a default breath signal as the final breath signal when the similarity score does not satisfy the similarity threshold;
provide the estimated breath signal as the final breath signal when the similarity score satisfies the similarity threshold.

5. The system as recited in claim 4, wherein the model breath signal and the default breath signal are respectively determined based on a plurality of previous estimated breath signals of the user stored in a memory.

6. The system as recited in claim 1,
wherein the one or more image sensors comprise a pair of stereo cameras; and
wherein the processing circuitry is further configured to determine disparity values between different portions of the image data obtained from the pair of stereo cameras, wherein the point cloud data is generated based on the disparity values.

7. A method, comprising:
performing, by a device comprising processing circuitry:
obtaining point cloud data of an environment that includes a user of the device for a period of time;
selecting one or more regions of interest in the point cloud data across the period of time, wherein the selected one or more regions of interest contain points in the point cloud data that correspond to upper-body landmarks of the user;
measuring respective motion of the points contained in the selected one or more regions of interest in the point cloud data over the period of time;
based on the respective motion of the points, generating an estimated breath signal of the user; and
providing a final breath signal based, at least in part, on the estimated breath signal.

8. The method as recited in claim 7,
wherein measuring the respective motion of the points contained in the selected one or more regions of interest in the point cloud data over the period of time, comprises:
dividing the one or more regions of interest into two or more smaller subregions;
independently measuring the respective motion of the points contained in the two or more subregions over the period of time;
wherein generating the estimated breath signal of the user, comprises:
based on the respective motion of the points in each of the two or more subregions, generating respective subregion breath signals; and
combining the subregion breath signals according to a weighting method to generate the estimated breath signal.

9. The method as recited in claim 7, wherein selecting the one or more regions of interest, comprises:
selecting a first reference point and a second reference point from the point cloud data corresponding to respective ones of the upper-body landmarks of the user as an upper boundary and lower boundary;
measuring a first distance from the first reference point to the second reference point;
selecting a third reference point and a fourth reference point corresponding to different ones of the upper-body landmarks of the user as a left boundary and a right boundary;
measuring a second distance from the third reference point to the fourth reference point;
determining height and width for the region of interest to create an area of interest based on the first distance and the second distance;
selecting a depth for the area of interest to create the region of interest, wherein the selection of the depth is such that when the region of interest is centered between the first and second reference points and the third and fourth reference points at least part of the points in the point cloud data corresponding to the upper-body landmarks of the user are contained within the region of interest.

10. The method as recited in claim 7, wherein providing the final breath signal, comprises:
comparing the estimated breath signal to a model breath signal to generate a similarity score between the estimated breath signal and the model breath signal; and
comparing the similarity score to a similarity threshold;
providing a default breath signal as the final breath signal responsive to determining that the similarity score does not satisfy the similarity threshold according to the comparing;
providing the estimated breath signal as the final breath signal responsive to determining that the similarity score satisfies the similarity threshold according to the comparing.

11. The method as recited in claim 7, wherein providing the final breath signal comprises sending the final breath signal to a different device over a wireless connection between the device and the different device, wherein the different device includes a display that provides a visualization based on the final breath signal.

12. A device, comprising:
a frame, configured to be worn on a head of a user;
two or more image sensors integrated in or coupled to the frame and configured to collect image data of at least an upper body of the user in an environment when the frame is worn on the head of the user over a period of time;
a controller for the device, comprising processing circuitry configured to:
obtain the image data;
generate, from the image data, point cloud data containing points representative of the user in the environment as captured in the image data over the period of time;
identify one or more regions of interest in the point cloud data encompassing points in the point cloud data that correspond to upper-body landmarks of the user;
measure respective motion of the points encompassed in the one or more regions of interest in the point cloud data over the period of time;
generate an estimated breath signal of the user; and
provide a final breath signal, based at least in part, on the estimated breath signal.

13. The device as recited in claim 12,
wherein the processing circuitry of the controller is further configured to convert the point cloud data from image coordinate system to a world coordinate system; and
wherein the identification of the one or more regions of interest in point cloud data from the transformed point cloud across the period of time, is based on the converted point cloud data in the world coordinate system.

14. The device as recited in claim 12, wherein to identify the one or more regions of interest, the processing circuitry of the controller is configured to:
select a first reference point and a second reference point from the point cloud data corresponding to respective ones of the upper-body landmarks of the user as an upper boundary and lower boundary;
measure a first distance from the first reference point to the second reference point;
select a third reference point and a fourth reference point corresponding to different ones of the upper-body landmarks of the user as a left boundary and a right boundary;
measure a second distance from the third reference point to the fourth reference point;
determine height and width for the one or more regions of interest to create an area of interest based on the first distance and the second distance;
select a depth for the area of interest to create the one or more regions of interest, wherein the depth is selected such that when the one or more regions of interest are centered between the first and second reference points and the third and fourth reference points at least part of the points in the point cloud data corresponding to the upper-body landmarks of the user are contained within the one or more regions of interest.

15. The device as recited in claim 12, wherein to identify the one or more regions of interest in the point cloud data across the period of time, the processing circuitry of the controller is configured to apply a trained machine learning model to identify the upper-body landmarks of the user.

16. The device as recited in claim 12, wherein to provide the final breath signal, the processing circuitry is configured to:
compare the estimated breath signal to a model breath signal to generate a similarity score between the estimated breath signal and the model breath signal stored in the device; and
compare the similarity score to a similarity threshold, where the processing circuitry of the controller is configured to:
provide a default breath signal as the final breath signal when the similarity score does not satisfy the similarity threshold;
provide the estimated breath signal as the final breath signal when the similarity score satisfies the similarity threshold.

17. The device as recited in claim 12, wherein the device further comprises a display visible to the user and wherein the final breath signal is provided to generate a visualization on the display corresponding to a breath cycle state of the user.

18. The device as recited in claim 12,
wherein the one or more image sensors comprise a pair of stereo cameras; and
wherein the processing circuitry of the controller is further configured to determine disparity values between different portions of the image data obtained from the pair of stereo cameras, wherein the point cloud data is generated based on the disparity values.

19. The device as recited in claim 12,
wherein the one or more image sensors comprise one or more depth sensors configured to collect depth data corresponding to the image data; and
wherein the point cloud data is generated based on the depth data.

20. The device as recited in claim 12, wherein the image data is obtained at the device from the one or more image sensors via a wireless communication.
